(19) United States
(12) Patent Application Publication
(10) Pub. No.: US 2012/0015023 A1
(43) Pub. Date: Jan. 19, 2012

(54) Treatment of tumors prostate with arsonoliposomes

(76) Inventors: Sophia G. Antimisiaris, Patras (GR); Panayiotis V. Ioannou, Petras (GR); Ming X. Wei, Chatou (FR)

(21) Appl. No.: 13/064,802

(22) Filed: Apr. 15, 2011

Related U.S. Application Data
(60) Provisional application No. 61/324,547, filed on Apr. 15, 2010.

(51) Int. Cl. A61K 9/127; A61P 35/00; A61K 31/285

(52) U.S. Cl. 424/450; 514/504

(57) ABSTRACT

The invention provides a method for treating a prostate proliferative disorder, such a prostate carcinoma or a begnin prostatic hyperplasia, in a patient, which method comprises administering arsonoliposomes comprising arsonolipids, wherein the arsonolipids are 2,3-diacyloxypropylarsonic acids, to a patient in need thereof.

The present application claims benefit of U.S. Provisional No. 61/324,547, filed Apr. 15, 2010, the entire contents of which is incorporated herein by reference.

The present invention relates to therapy of a proliferative disorder of prostate, in particular prostate cancer.

### TECHNICAL BACKGROUND

The walnut-sized prostate is an encapsulated organ of the mammalian male urogenital system. Located at the base of the bladder, the prostate is partitioned into zones referred to as the central, peripheral and transitional zones, all of which surround urethra. Histologically, prostate is a highly microvascularized gland comprising fairly large glandular spaces lined with epithelium which, along with the seminal vesicles, supply the majority of fluid to the male ejaculate.

The major neoplastic disorders of the prostate are benign enlargement of the prostate, also called benign prostatic hyperplasia (BPH), and prostatic carcinoma, a type of neoplasia. BPH is very common in men over the age of fifty. It is characterized by the presence of a number of large distinct nodules in the periurethral area of the prostate. Although benign and not malignant, these nodules can produce obstruction of the urethra causing nocturia, hesitancy to void, and difficulty in starting and stopping a urine stream upon voiding the bladder. Left untreated, a percentage of these prostate hyperplasias and neoplasias may develop into malignant prostatic carcinoma.

Prostate cancer is one of the most common causes of cancer deaths in males. It accounts for about 40% of all cancers diagnosed in men.

In its more aggressive form, malignant transformed prostatic tissues escape from the prostate capsule and metastasize invading locally and throughout the bloodstream and lymphatic system. Metastasis, defined as tumor implants which are discontinuous with the primary tumor, can occur through direct seeding, lymphatic spread and hematogenous spread. All three routes have been found to occur with prostatic carcinoma. Local invasion typically involves the seminal vesicles, the base of the urinary bladder, and the urethra. Direct seeding occurs when a malignant neoplasm penetrates a natural open field such as the peritoneal, pleural or pericardial cavities. Cells seed along the surfaces of various organs and tissues within the cavity or can simply fill the cavity spaces. Hematogenous spread is typical of sarcomas and carcinomas. Hematogenous spread of prostatic carcinoma occurs primarily to the bones, but can include massive visceral invasion as well. It has been estimated that about 60% of newly diagnosed prostate cancer patients will have metastases at the time of initial diagnosis.

Surgery or radiotherapy is the treatment of choice for early prostatic neoplasia. Surgery involves complete removal of the entire prostate (radical prostatectomy), and often removal of the surrounding lymph nodes, or lymphadenectomy. Radiotherapy, occasionally used as adjuvant therapy, may be either external or interstitial using 125I. Endocrine therapy is the treatment of choice for more advanced forms. The aim of this therapy is to deprive the prostate cells, and presumably the transformed prostate cells as well, of testosterone. This is accomplished by orchiectomy (castration) or administration of estrogens or synthetic hormones which are agonists of luteinizing hormone-releasing hormone. These cellular messengers directly inhibit testicular and organ synthesis and suppress luteinizing hormone secretion which in turn leads to reduced testosterone secretion by the testes. In normal prostate, removal of androgenic hormones results in regression of the gland involving apoptosis of more than 60% of the luminal epithelial cells. Although often initially sensitive to removal of androgens, prostate cancer cells eventually lose this response and continue to grow and spread even in the absence of androgenic steroids. Despite the advances made in achieving a pharmacologic orchiectomy, the survival rates for those with late stage carcinomas are low.

Current therapeutic regimens for metastatic disease typically involve both chemical and surgical androgen ablation, which although has been demonstrated to extend life when compared to untreated patients, almost invariably results in the development of hormone-refractory disease and the demise of the patient.

In view of the shortcoming of existing therapies, there exists a need for improved modalities for treating prostate proliferative disorders.

### SUMMARY OF THE INVENTION

The inventors have shown that arsonoliposomes comprising arsonolipids, wherein the arsonolipids are 2,3-diacyloxypropylarsonic acids, are highly specific and efficient to target and inhibit proliferation of prostate tumor cells in vitro and in vivo.

On this basis, the invention provides a method for treating a prostate proliferative disorder, such as a prostate carcinoma or a begnin prostatic hyperplasia, in a patient, which method comprises administering arsonoliposomes comprising arsonolipids, wherein the arsonolipids are 2,3-diacyloxypropylarsonic acids, to a patient in need thereof.

### LEGENDS TO THE DRAWINGS

FIG. 1 is a graph that shows the effect of arsonoliposomes on the viability of PC3 (prostate cancer cells). Cells were incubated with various concentrations of arsonoliposomes for 24 h. (A) Arsonoliposomes containing 27 mol % arsonolipids (Ars) were evaluated. (B) Arsonoliposomes containing 10 mol % Ars were evaluated. Results are expressed as viability (percent of viable cells in comparison with the control cells) versus arsonolipid content of each liposomal formulation. Each experiment was performed in triplicate, and the bars represent SD values.

FIG. 2 is a graph that shows mice body weight over the time. Group 1: Vehicle (PBS). Group 2: Positive control group. Animals were treated with a standard chemotherapeutic agent (docetaxel, marketed as Taxotere®) at a dose of 12.5 mg/kg. Group 3: Test group. Animals were treated with the arsonoliposomes at a dose of 4× diluted test arsonoliposomes Group 4: Test group. Animals were treated with the arsonoliposomes at a dose of 2× diluted test arsonoliposomes. Group 5: Test group. Animals were treated with the arsonoliposomes at a dose of non-diluted test arsonoliposomes (i.e. 20 mg/ml of total lipid which is equivalent to 7.33 mg/ml of arsonolipid).

FIG. 3 is a graph that shows mice tumor volume over the time, in each group.

FIG. 4 is a photograph that shows a mouse from control group 1, 54 days after implantation of PC-3 cells.

FIG. 5 is a photograph that shows a mouse from group 5 (treatment with concentrated arsonoliposomes), 54 days after implantation of PC-3 cells.

FIG. 6 shows a representative tumor section from a mouse of group 1, after hematoxylin and easin staining, at day 7 post-implantation.

FIG. 7 shows a representative tumor section from a mouse of group 3, after hematoxylin and easin staining, at day 7 post-implantation. Large necrosis area was observed in the center of this section.

FIGS. 8A and 8B show representative tumor sections from a mouse of group 5, after hematoxylin and easin (H&E) staining, at day 74 post-tumor implantation. On FIG. 8A, the arrows show a necrotic area with an infiltration of lymphocytes. On FIG. 8B, the circle shows a massive necrotic area.

### DETAILED DESCRIPTION OF THE INVENTION

As used in the present invention, the term <<arsonoliposomes>> refers to liposomes prepared using arsonolipids. Arsonolipids are arsenic-containing analogues of phosphonolipids as described in Tsivgoulis et al, Phosphorus, Sulfur and Silicon, 1991, 57:189-193, and Tsivgoulis et al, Phosphorus, Sulfur and Silicon, 1991, 63:329-334.

The arsonolipids useful in the present invention are 2,3-diacyloxypropylarsonic acids, i.e. arsenic-containing analogues of phosphonolipids with two acyl chains.

The arsonolipids useful in the present invention may be racemic 2,3-diacyloxypropylarsonic acids or optically active (R or S) 2,3-diacyloxypropylarsonic acids.

Preferred arsonolipids have formula I:

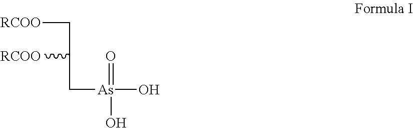

wherein the RCOO acyl chains are saturated linear, saturated branched, unsaturated linear or unsaturated branched acyl chains presenting from 6 to 20 carbon atoms, preferably from 8 to 18, preferably from 10 to 18, even more preferably from 12 to 18 carbon atoms. The acyl chains are preferably chosen among hexanoyl, octanoyl, decanoyl, dodecanoyl, tetradecanoyl, hexadecanoyl and octadecanoyl chains. The highly preferred acyl chain is the palmitoyl chain (containing 16 carbon atoms, i.e, wherein R is C15H31).

The arsonoliposomes useful in the present invention may further comprise a sterol, such as cholesterol molecules, and/or 1,2-distearoyl-sn-glycero-3-phosphocholine (DSPC) molecules, having formula II

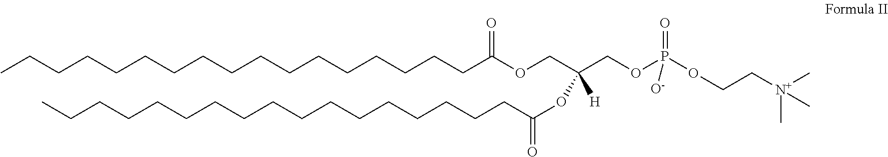

Cholesterol and DSPC molecules are included in the membrane of the arsonoliposomes.

Preferred arsonoliposomes contain DSPC, arsonolipids and cholesterol. DSPC/arsonolipid/cholesterol molar ratio in the arsonoliposomes preferably range from 10/10/10 to 18/2/10 and is preferably 12/8/10—equivalent to 27 mol % arsonolipid—or 17/3/10—equivalent to 10 mol % arsonolipid.

In a particular embodiment, the arsonoliposomes may be PEGylated, i.e. they carry polyethylene glycol (PEG) molecules.

PEG molecules may be included by various means. For instance, they may be conjugated to phospholipids, such as 2-distearoyl-sn-glycero-3-phosphoethanolamine (DSPE), dipalmitoyl phosphatidylethanolamine (DPPE), or dioleyl-sn-glycero-3-phosphoethanolamine (DOPE), or mixtures thereof.

In a preferred embodiment, 2-distearoyl-sn-glycero-3-phosphoethanolamine (DSPE) molecules are conjugated to polyethylene glycol (PEG) and included in the arsonoliposomes to provide vesicle surface PEG-ylation.

The molecular weight of PEG molecules may range from 300 g/mol to 10 000 000 g/mol. and is preferably between 500 g/mol and 5000 g/mol. In a preferred embodiment, PEG molecules conjugated to DSPE molecules are PEG 2000 molecules—i.e. PEG molecules with a molecular weight of 2000 g/mol. DSPE molecules (e.g. 2-10 mol %) are PEG-ylated with PEG 2000, as shown e.g. in formula III

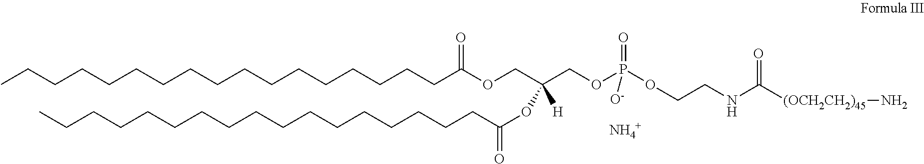

In a preferred embodiment, the arsonoliposomes thus further comprise 1,2-distearoyl-sn-glycero-3-phosphoethanolanine (DSPE) molecules conjugated with polyoxyethylene glycol (PEG) 2000.

In a preferred embodiment, the arsonoliposomes contain PEG-conjugated phospholipids which represent from 2 to 20 mol %, preferably 5 to 10%, of the arsonoliposome lipids, preferably 8 mol %.

Suitable arsonoliposomes for use in this invention include large unilamellar vesicles (LUVs), multilamellar vesicles (MLVs), small unilamellar vesicles (SUVs) and interdigitating fusion liposomes, most preferably they are small unilamellar vesicles (SUVs).

Arsonoliposomes used in the present invention can be prepared as described previously, e.g. in Fatouros et al. Chem Phys Lipids 2001 109, 75-89, using the one step method of Talsma et al, 1994, J. Pharm. Sci, 83: 276-280. In brief, arsonolipids (after removing the organic solvents) are mixed with 5 mM phosphate (or other) buffer (pH 7.4), and 20 mM NaCl, and magnetically stirred for 4 h at 70-80° C. After formation of liposomes, the samples are left to anneal for at least 1 h. In order to reduce liposome size, the large liposome suspension initially produced is subjected to probe sonication. Following sonication, the liposome suspensions are left to stand for about 2 hours at 65° C. Titanium fragments or any multilamellar vesicles or liposomal aggregates can be removed by centrifugation at 10,000 rpm (˜1,500 g) for 10 min.

The arsonoliposomes are useful for treating a prostate proliferative disorder.

The term “prostate proliferative disorder” means any abnormal proliferation of prostatic cells, including begnin hyperplasia or tumor, and cancerous tumors. In a particular embodiment, this disorder is benign prostatic hyperplasia (BPH). In another embodiment, the disorder is prostate cancer, at any stage. The arsonoliposomes are particularly useful for treating metastatic prostate carcinoma.

Prostate cancer has four basic stages which are:

Stage 1—the cancer is very small and completely inside the prostate gland which feels normal during a rectal examination
Stage 2—the cancer is still inside the prostate gland, but is larger and a lump or hard area can be felt during a rectal examination
Stage 3—the cancer has broken through the covering of the prostate and may have grown into the tubes which carry semen
Stage 4—the cancer has grown into the bladder or rectum, or has spread to the lymph nodes or another part of the body, such as the bones, liver or lungs e.g. stage IV prostate carcinoma.

More particularly, treatment with the arsonoliposomes may be useful in patients with stage IV prostate carcinoma.

The term “patient” refers to a human or non human male mammal, especially a man.

As used herein, the term “treatment” or “therapy” includes curative and/or prophylactic treatment. More particularly, curative treatment refers to any of the alleviation, amelioration and/or elimination, reduction and/or stabilization (e.g., failure to progress to more advanced stages) of a symptom, as well as delay in progression of a symptom of a particular disorder.

Prophylactic treatment refers to any of: halting the onset, reducing the risk of development, reducing the incidence, delaying the onset, reducing the development, as well as increasing the time to onset of symptoms of the disorder.

The arsonoliposomes in the present invention are preferably prepared as sonicated suspensions. The arsonoliposomes may have a vesicle mean diameter of greater than 20 nm, or from about 20 to about 500 or 1000 nm, preferably from about 50 to about 150 nm, still more preferably from about 70 to about 120 nm.

Preferably, arsonoliposomes of the invention contain a sterol, such as cholesterol. The presence of 30 mol % of cholesterol significantly modifies the phase transition characteristics of the lipids and incorporating cholesterol permit membrane-rigidifying.

Arsonoliposomes useful in the invention may also contain therapeutic lipids, which examples include ether lipids, phosphatidic acid, phosphonates, ceramide and ceramide analogs, sphingosine and sphingosine analogs and serine-containing lipids.

The present invention may further include a chemical moiety attached to the membrane of the arsonoliposome. The chemical moiety may be for targeting, stabilizing or protecting the arsonoliposome construct. As used herein the term “attached” relates to a direct or indirect, covalent or non-covalent bond and connection, respectively, between a chemical moiety and another component of the arsonoliposome. Examples of chemical moiety include biotin-streptavidin, amino-reactive groups (e.g. carbodiim ides, hydroxylmethylphosphine, imidoester, N-hydroxysuccinimide esters, isothiocyanates, isocyanates), sulfhydryl-reactive groups apart from the AsO₃H₂ (e.g. maleimides, haloacetyls, pyridyl disulfides, aziridines) carboxyl-reactive molecules (e.g. carbodiimides, carbodiimidazole), hydroxyl-reactive groups (e.g. carbonyldiimidazole, alkyl halides, isocyanates), or can include a stabilizing moiety for increasing the circulation time of the arsonoliposome once it is administered, such as ganglioside GM1, phosphatidylinositol or polyethylene glycol (PEG), e.g., PEGs having a molecular mass between about 1,000 and about 10,000 g/mol. Targeting moieties may also include detergents, proteins, and peptides, such as an antibody or fragment thereof, a single-chain antibody or fragment thereof, a receptor ligand or fragment thereof; a carbohydrate; or a ligand.

The arsonoliposomes preferably act as anti-tumor agents by themselves, i.e. when they do not carry any further anti-tumor drug. In another embodiment, they may contain an anti-tumor drug which they deliver to the tumor cells.

The drug may be loaded into arsonoliposomes using both passive and active loading methods. Passive methods of encapsulating active agents in liposomes involve encapsulating the agent during the preparation of the liposomes. This includes a passive entrapment method described by Bangham, et al. (J. Mol. Biol. (1965) 12: 238). This technique results in the formation of multilamellar vesicles (MLYs) that can be converted to large unilamellar vesicles (LUVs) or small unilamellar vesicles (SUVs) upon extrusion. Additional suitable methods of passive encapsulation include an ether injection technique and the Reverse Phase Evaporation technique. Active methods of encapsulation include the pH gradient loading technique described in U.S. Pat. Nos. 5,616,341, 5,736,155 and 5,785,987 and active metal-loading. A preferred method of pH gradient loading is the citrate-base loading method utilizing citrate as the internal buffer at a pH of 4.0 and a neutral exterior buffer.

The arsonoliposomes may be administered with pharmaceutically acceptable excipients, by any convenient route including intravenous, transdermal, subcutaneous, mucosal, intramuscular route. A preferred route is the subcutaneous or the intramuscular route. In another embodiment, the arsonoliposomes are administered in situ, directly into or in vicinity to the tumor cells, e.g. into the prostate or the cavernous body, if needed.

The arsonoliposomes can be formulated into preparations for injection by dissolving, suspending or emulsifying them in an aqueous solvents; and if desired, with conventional additives such as isotonic agents, suspending agents, emulsifying agents, stabilizers and preservatives so long as such preparations do not compromise the liposomal structure. For instance, pharmaceutical compositions comprising arsonoliposomes may further comprise pharmaceutically acceptable excipients, including water, buffered water, 0.9% saline, 0.3% glycine, 5% dextrose and the like, including glycoproteins for enhanced stability, such as albumin, lipoprotein, globulin, and the like and cryoprotectants, if needed, such as trehalose, sucrose or maltose and the like. These compositions may be sterilized by conventional, well-known sterilization techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and the like.

Additionally, the pharmaceutical composition may include lipid-protective agents which protect lipids against free-radical and lipid-peroxidative damages on storage. Lipophilic free-radical quenchers, such as alpha-tocopherol and water-soluble iron-specific chelators, such as ferrioxamine, are suitable.

The concentration of arsonoliposomes in the pharmaceutical compositions can vary widely, such as from less than about 0.05%, usually at or at least about 2-5% to as much as 10 to 30% by weight and will be selected primarily by fluid volumes, viscosities, and the like, in accordance with the particular mode of administration selected. For example, the concentration may be increased to lower the fluid load associated with treatment.

The appropriate dose to be administered depends on the subject to be treated, such as the general health of the subject, the age of the subject, the state of the disease or condition, the weight of the subject, etc.

The liposome construct may be administered to a man in a dose of an effective amount of from about 1-10-200 mg of arsonoliposomes (total lipid)/kilogram of body weight which corresponds to 1.75-35 mg of arsonolipid/kilogram of body weight. The dosage amount may depend on the size of the arsonoliposomes.

The frequency of administration of the composition of arsonoliposomes, as with the doses, is determined by the medical practitioner based on age, weight, disease status, health status and patient responsiveness. In a particular embodiment, the composition is administered once or twice a week, preferably for a period of at least one month.

The following examples illustrate the invention without limiting its scope:

### EXAMPLES

### Example 1

### In Vitro Data on Prostate Tumor Cell Line

Materials and methods

### Reagents

Phosphatidyl choline (PC) (from egg, grade 1), 1,2-distearoyl-sn-glycero-3-phosphocholine (DSPC) (synthetic, grade 1) and 1,2-distearoyl-sn-glycero-3-phosphoethanolanine conjugated to PEG (MW 2000) (DSPE-PEG2000) (synthetic, grade 1) were purchased from Avanti Polar Lipids. Cholesterol (Choi) (pure) and Triton X-100 were obtained from Sigma-Aldrich ((O.M.), Athens, Greece). The water used was deionized and then distilled. All other reagents and solvents used throughout the study were of analytical grade and were purchased from Sigma-Aldrich. All media used for cell growth and handling as well as fetal calf serum (FCS) were purchased from Biochrom (Berlin, Germany), and were of cell culture grade. The rac-arsonolipid (C16) (Ars) (2,3-dipalmitoyloxypropylarsonic acid) with a palmitic side chain (R═C15H31 in formula I) was synthesized and characterized, as described in detail before (Tsivgoulis et al, 1991, Phosphorus, Sulfur and Silicon, 57:189-193, and Tsivgoulis et al, 1991, Phosphorus, Sulfur and Silicon, 63:329-334).

### Cell Line

Prostatic cancer (PC3) cells (obtained from ATCC, USA) were cultured in HAM's F-10 supplemented with 10% foetal calf serum (FCS) and used at passages 19-21. The medium also contained 100 IU/mL penicillin and 10 μg/mL streptomycin and cultures were maintained at 37° C., 5% CO₂ and 100% humidity.

### Arsonoliposome Composition

Using Ars, PC or DSPC, Chol and in some cases DSPE-PEG, liposomes having the following lipid compositions were prepared: (i) PC-based arsonoliposomes (PC/Ars/Chol 12:8:10 by mole, with 27 mol % arsonolipid content and in some cases PC/Ars/Chol 17:3:10 by mol, with

10 mol % Ars content); nonPEGylated and PEGylated (in which 8 mol % DSPE-PEG2000 lipid was incorporated). And (ii) DSPC-based arsonoliposomes (DSPC/Ars/Chol 12:8:10 by mol, with 27 mol % Ars content and in some cases DSPC/Ars/Chol 17:3:10 by mol, with 10 mol % Ars content); nonPEGylated and PEGylated (in which 8 mol % DSPE-PEG2000 lipid was incorporated).

### Preparation of Arsonoliposomes

Arsonolipid (Ars)-containing liposomes were prepared as described previously (Fatouros et al. Chem Phys Lipids 2001 109, 75-89, Fatouros et al, J. Nanosci. Nanotechnol, 2006, 6, 2618-2687). In brief, lipids (after removing the organic solvents with a nitrogen stream) were mixed with 5 mM phosphate buffer (pH 7.4) and 20 mM NaCl and magnetically stirred vigorously on a hot plate for 4 h at 70-80° C. After formation of liposomes, the samples were left to anneal for at least 1 h at the liposome preparation temperature.

In order to reduce liposome size, the large liposome suspension initially produced was sonicated, using a Vibra-cell probe sonicator (Sonics and Materials, UK) equipped with a tapered microtip, for at least two 5 min cycles. In all cases the initially turbid liposomal suspension was clear (transparent) after sonication. Following sonication, the liposome suspensions were left to stand for 1 h at 65° C. (or higher than the transition temperature of the lipids used in each case), in order to anneal any structural defects. The titanium fragments and any multilamellar vesicles or liposomal aggregates were removed by centrifugation at 10,000 rpm (=1500×g) for 10 min.

The lipid content of the samples was routinely determined using a colorimetric technique, which is widely applied for phospholipids, the Stewart assay, in which the ability of phospholipids to form a complex with ammonium ferrothiocyanate in organic solution is utilized (Stewart, 1980, Anal. Biochem, 104:10-14). This assay was found to also detect Ars (at the high concentrations of the initial dispersions). In brief, liposome samples (20 μL) are vortexed with 2 mL of a solution of ammonium ferrothiocyanate (0.1 M) and 2 mL of chloroform. The OD-485 nm of the chloroform phase is measured and the lipid concentration of samples is calculated by comparison with a calibration curve. The exact arsenic content of all arsonoliposome preparations was determined by graphite furnace atomic absorption spectroscopy (GFAAS), as previously described (Desaulniers et al, 1985, At. Spectrosc, 6:125-127; Devalla et al, 2003, Appl. Organomet. Chem., 17:906-912)). In brief, 20 μL of arsonoliposomes were digested with nitric acid at 80° C. and after this the residue was dissolved in a mixture of 0.5 mL 0.4% v/v nitric acid and 0.5 mL of H₂O₂.

These samples were kept at 4° C. until GFAAS analysis.

Liposome samples were kept at 4° C. until use in cell culture experiments.

### Characterization of Arsonoliposome (Size, Zeta Potential and Integrity)

The liposomes prepared were characterized by measuring their size by DLS (dynamic light spectroscopy) with a Malvern Zetasizer 5000 (Malvern, UK), as described before (Fatouros et al. Chem Phys Lipids 2001 109, 75-89; Zagana et al, Int. J. Pharma, 2008, 347:86-92; Zagana et al, Biomed. Pharmacother, 2007, 61:499-504). In brief, liposome dispersions were diluted with filtered PBS pH 7.40 and sized immediately. The arsonoliposome electrophoretic mobility was also measured at 25° C. (Zetasizer 5000 Malvern Instruments), after diluting the vesicle dispersion with filtered PBS pH 7.40. Zeta potentials of the dispersions were calculated (by application of the Helmholtz-Smolowkovski equation). Measurement of arsonoliposome integrity: For some arsonoliposome types, vesicle integrity was evaluated by measuring the latency of vesicle encapsulated calcein (Kokkona et al, 2000, Eur. J. Pharm. Sci., 9:245-252).

Calcein was encapsulated in the vesicles (during their preparation as described above) at a quenched concentration (100 mM) and the vesicles were incubated in PBS buffer or in presence of serum proteins (80% v/v FCS) for periods up to 24 h at 37° C. and under mild agitation (30 rpm). For calcein latency calculation, samples from the incubates were drawn (20 ul) and diluted with 4 mL PBS, pH 7.40, and fluorescence intensity (FI) was measured (EM 470 nm, EX 520 nm) by a Shimatzu RF1000 Spectrofluorimeter, before and after addition of Triton X-100 at a final concentration of 1% v/v (that ensures liposome disruption and release of all encapsulated dye). Percent latency (% latency) was calculated from the following equation:

% Latency=((1.1F_{AT}−F_{BT})/1.1F_{AT})×100

where, F_{BT} and F_{AT} are calcein FIs before and after the addition of Triton X-100, respectively.

### Cell Viability Assays

For cell viability assays, cells were seeded at an initial concentration of 1×10⁵ cells/mL in 24-well tissue culture plates, and incubated in medium with or without arsonoliposomes for periods of 24 h. Cell viability after incubations was assessed by: (i) tryptan blue exclusion using a hemocytometer, and (ii) by measuring the number of cells, using the 3-(4,5-dimethylthiazol-2-yl)-2,5-dimethyltetrazolium bromide (MU) assay (Mosman et al, Immunol. Methods 1983, 65:55-63). For this, MU stock (5 mg/ml in phosphate buffer saline PBS) at a volume equal to one-tenth of the volume of medium was added to all wells and plates were incubated at 37° C. for 2 h. After incubation, the medium was removed, the cells were washed with PBS pH 7.4 and 100 μL of acidified isopropanol (0.33 mL HCL in 100 mL isopropanol) was added to all wells and agitated thoroughly to solubilise the dark blue formazan crystals. The solution was transferred to a 96-well plate and immediately read on a microplate reader (BioRad) at a wave-length of 490 nm.

The effect of arsonoliposomes on cell viability was assessed by comparing the number of live cells in treated cells with those in the control cells in which plain buffer but no arsonoliposomes were added. Growth inhibition concentrations (50%, IC 50) were calculated from interpolations of the graphical data. Additional controls were performed, in which the effect of sonicated conventional phospholipid liposomes PC/Chol (2:1 mol/mol) on the cell viability was evaluated under identical experimental conditions (lipid concentration, time of exposure, etc.). In all cases PC/Chol liposomes did not demonstrate any cytotoxic effects.

### Results:

### Arsonoliposome Physicochemical Properties

The arsonoliposome mean diameter ranged between 79.9 and 112.5 nm, depending on the arsonoliposome type (Table 1). PEGylation results in significant (at p=0.05) increase in the mean diametre of arsonoliposomes as also reported before. Additionally, arsonolipids give a negative surface charge to vesicles (Table 1), while this charge is abolished when PEG molecules are present on the arsonoliposome surface, proving that the surfaces of arsonoliposomes are indeed coated with PEG molecules.

TABLE 1
Physicochemical properties of the arsonoliposomes

| | Vesicle mean | |
|---|---|---|
| | diameter | Zeta-potential |
| Arsonoliposome (lipid composition) | (nm)^{a)} | (mV)^{a)} |
| PC-based arsonoliposomes (27%)b) | 82.55 (0.45) | −20.45 (0.22) |
| (PC/Ars/Chol (12:8:10)) | | |
| PEGylated PC (27%) (PC/Ars/Chol | 94.00 (0.80) | 0.8 (2.1) |
| (12:8:10) + 8 mol % DSPE-PEG2000) | | |
| DSPC-based arsonoliposomes (27%) | 79.9 (2.9) | −23.2 (1.4) |
| (DSPC/Ars/Chol (12:8:10)) | | |
| PEGylated DSPC (27%) (DSPC/Ars/Chol | 112.5 (1.7) | 0.3 (1.2) |
| (12:8:10) + 8 mol % DSPE-PEG2000) | | |

These measurements were performed in PBS buffer, pH 7.4, at 25° C. The corresponding values of PC-based arsonoliposomes are also stated, for direct comparison.
^{a)}All values are mean values measured from at least three different batches of liposomes, and SD of each mean is reported.
b)The value in parenthesis is the mol % content of arsonoliposomes in Ars.

The integrity of PEGylated PC arsonoliposomes was evaluated by measuring the release of vesicle-encapsulated calcein, during incubation of the vesicles in buffer or in presence of serum proteins (80% FCS), for 24 h, as studied before for PEGylated (and non-PEGylated) DSPC arsonoliposomes (Piperoudi et al, Chem. Phys. Lipids, 2006, 139:96-106). The integrity of non-PEGylated PC arsonoliposomes was also evaluated under identical conditions for comparison.

Stability of the arsonoliposomes was shown to depend on their structure, according to the following rank order: PEGylated DSPC arsonoliposomes are more stable than DSPC-based arsonoliposomes, which are more stable than PEGylated PC arsonoliposomes, which are more stable than PC-based arsonoliposomes.

### Arsonoliposomes Induced Inhibition of Prostate Cancer Cell Growth:

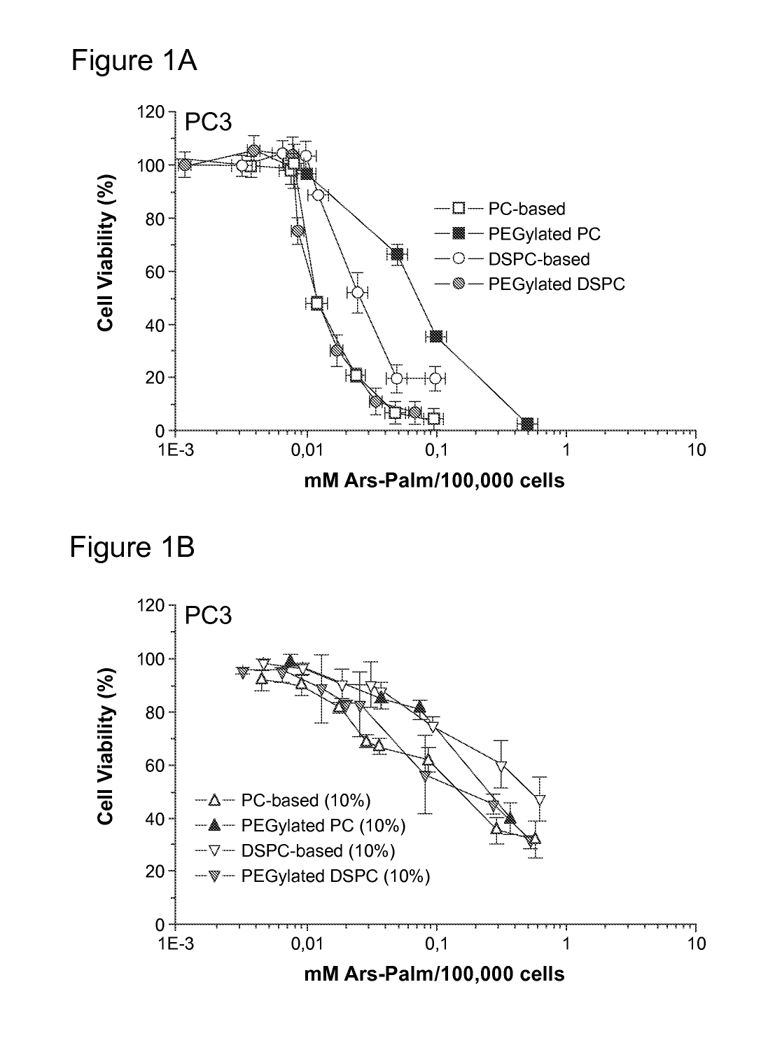

The effect of increasing concentrations of arsonoliposomes (expressed as arsonolipid Ars concentration) on the viability of the cells (after 24 h of co-incubation) is presented in FIG. 1. IC50 values for all cell types studied were calculated (for each type of arsonoliposome studied) and are listed in Table 2.

TABLE 2
Growth-inhibitory concentrations (50%, IC50) of arsonoliposomes
(expressed as the Ars content of arsonoliposomes in each case, Ars
(mM)/10⁵ PC-3 cells)

| Arsonoliposome composition | IC50^{a)} (Ars (mM)/10⁵ PC-3 cells) |
|---|---|
| PC-based (27%)^{b)} | .0117 (.0024) |
| PC-based (10%) | .172 (.010) |
| ^{c)}PEGylated PC (27%) | .076 (.013) |
| PEGylated PC (10%) | .293 (.043) |
| DSPC-based (27%) | .0262 (.0047) |
| DSPC-based (10%) | .544 (.011) |
| PEGylated DSPC (27%) | .0132 (.0013) |
| PEGylated DSPC (10%) | .1804 (.0034) |

^{a)}The values were calculated from interpolations of the graphical data presented in FIG. 1 using the Microcal Origin Program (Version 5). SD values for the IC50's (presented in brackets) were calculated as the mean of the SD's from the two closer measured viability values for each case.
^{b)}The numbers in parenthesis denote the mol % content of Ars, in each arsonoliposome type (27% means phospholipid/Ars/Chol 12/8/10, and 10% means phosholipid/Ars/Chol 17/3/10).
^{c)}PEGylated PC arsonoliposomes contain also 8 mol % DSPE-PEG2000.

### Example 2

### In Vivo Data on Prostate Tumor Mouse Model

Materials and methods

### Arsonoliposomes

The arsonoliposomes tested in this experiment were PEGylated DSPC based-arsonoliposomes having DSPC/Ars/Chol in proportions 12/8/10+8 mol % DSPE-PEG2000. with 27 mol % Ars content.

### Cell Culture

Log-growing human prostate tumor cells, PC-3 (grade IV prostate adenocarcinoma, from bone metastasis-obtained from ATCC, # CRL1470), were trypsinized, counted, washed and resuspended in serum-free DMEM medium for sub-cutaneous (S.C.) injection (in the organ, prostate) onto nu/nu athymic mice.

3.0×10⁶ cells per mouse in 200 μl were injected to nu/nu athymic mice (Balb/C nude; Harlan, France) Mice were 5-6 weeks old males, of about 20 g each.

### Animal Groups in Designed Experiment

### Experimental Design:

Group 1: Vehicle (PBS). Treatment regimen: twice a week for 2 weeks, I.P.
Group 2: Positive control group. Animals were treated with a standard chemotherapeutic agent (docetaxel, marketed as Taxotere®) at a dose of 12.5 mg/kg. Treatment regimen: twice at day 10 and day 20, I.P.
Group 3: Test group. Animals were treated with the arsonoliposomes at a dose of 4× diluted test arsonoliposomes (i.e. 5 mg/ml of total lipid which is equivalent to 1.83 mg/ml of arsonolipid (Ars)). Treatment regimen: twice a week for two weeks, I.P.
Group 4: Test group. Animals were treated with the arsonoliposomes at a dose of 2× diluted test arsonoliposomes (i.e. 10 mg/ml of total lipid which is equivalent to 3.66 mg/ml of arsonolipid (Ars). Treatment regimen: twice a week for two weeks, I.P.
Group 5: Test group. Animals were treated with the arsonoliposomes at a dose of non-diluted test arsonoliposomes of 20 mg/ml of total lipid which is equivalent to 7.33 mg/ml of arsonolipid (Ars). Treatment regimen: twice a week for two weeks, I.P.

Mice body weight evolution for monitoring systemic toxicity

The mice body weights were regularly measured during the entire study.

Anti-tumor activity in mice bearing prostate tumors

The inhibition of tumor growth was measured (tumor length, width and volume) twice a week from day 0 (tumor cell implantation) by using an external caliper. The average tumor volume (Mean+SD; Mean+SEM) was calculated by using the following formula: V=Length×Width2/2.

### Histological Analysis on Tumors

At the end of experiment, the animals were anesthetized by using a mixture of Ketamine/Xylazine and then sacrificed by an overdose of Ketamine/Xylazine. For all mice of each group: primary tumors were removed and carefully analyzed. For each tumor, it was fixed with 4% Formalin and paraffin embedded for histological analysis and later for hematoxylin and eosin staining.

### Results

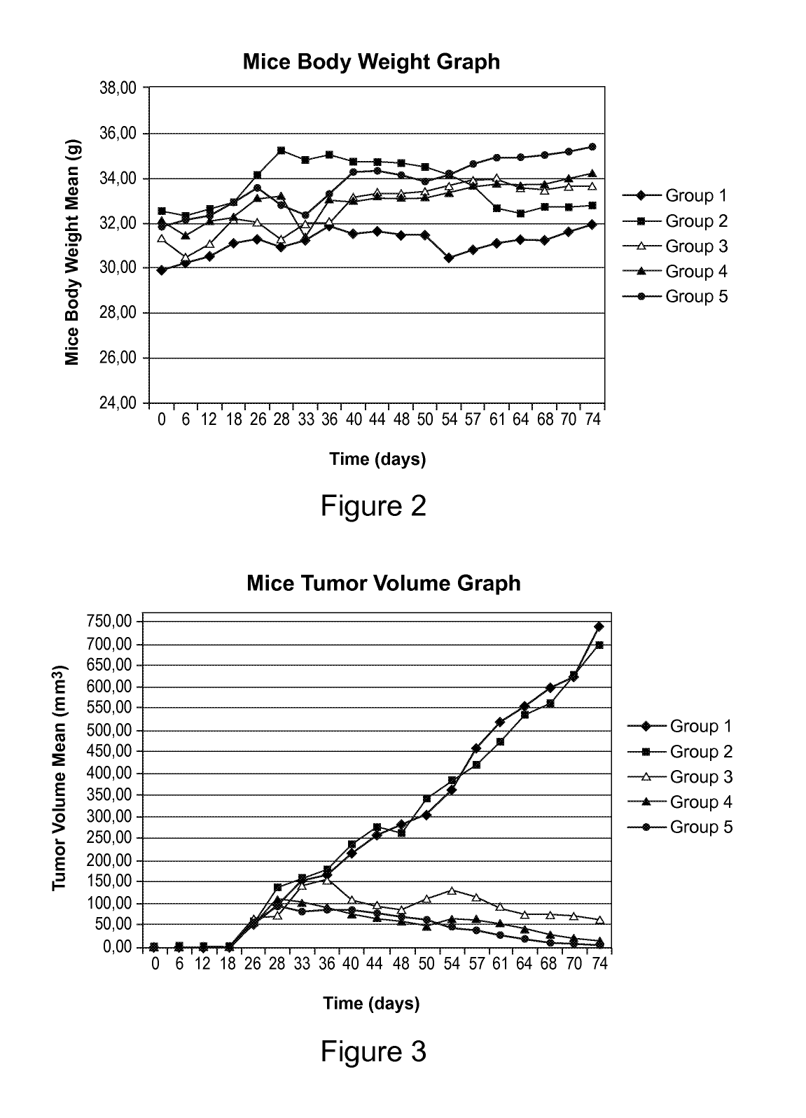

FIG. 2 shows that the test arsonoliposomes did not induce systemic toxicity in mice even with highest concentration.

FIG. 3 shows evolutions of mice tumor volume. Tumors in control group (group 1, treated with 1×PBS) were steadily growing and reached an average volume of 750 mm³. Whereas, tumors in treated groups (three doses in group 3, 4 and 5) were dramatically inhibited and the tumor volumes were observed between 4 mm³-62 mm³. This observed tumor inhibition seems to act in a dose-dependent manner. In two groups (groups 4 and 5) with high concentrations of arsonoliposomes, two mice in each group became tumor-free at day 74 post-treatment.

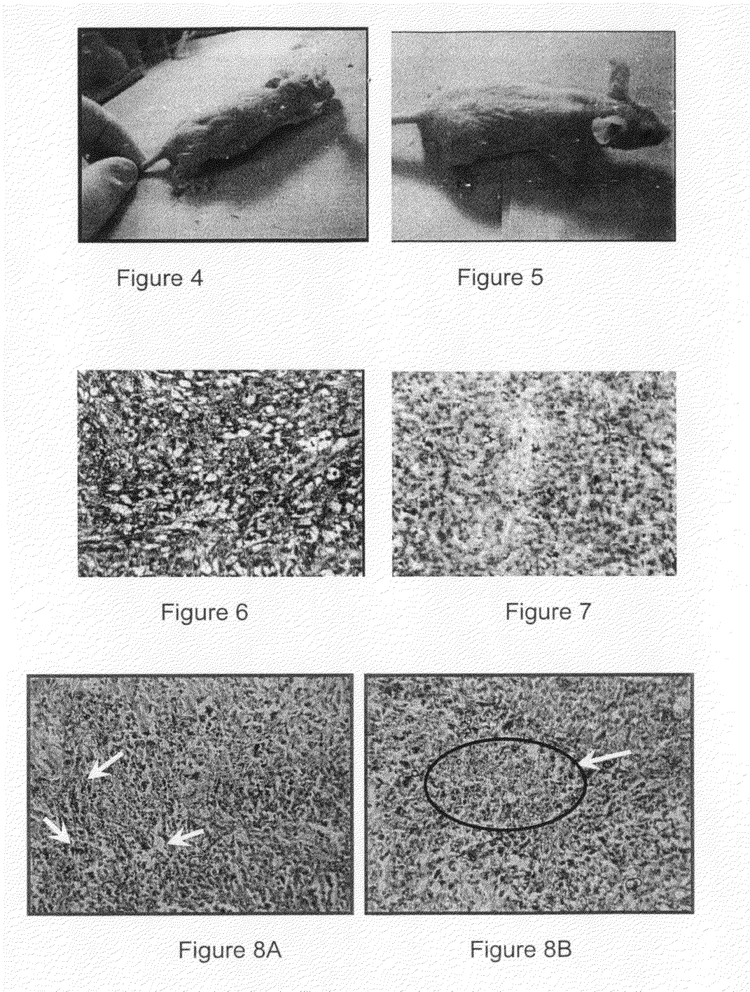

This strong tumor inhibition can be also visualized in photographs (see FIGS. 4 and 5). The tumors in control group can be physically “palpated” and seen with naked eyes. However, the “tumors” in treated group can be hardly seen and real tissue natures have to be confirmed by histological analysis.

Histological studies gave the following results: a number of necrotic areas were observed in tumor sections only in arsonoliposome-treated groups (see FIGS. 6, 7, 8A and 8B).

As a conclusion, the arsonoliposomes showed an extremely strong anti-tumor effect in experimental prostate cancer model. A significant tumor regression, and even a total regression of tumors in certain mice in groups 4 and 5 have been observed, in comparison with control group treated with PBS.

Furthermore, angiogenesis was reduced in the tumor in two arsonoliposome-treated groups, as evidenced by the measure of Interstitial Fluid Pressure (IFP).

TABLE 3
Interstitial Fluid Pressure (IFP)

| IFP in Control mice | IFP in arsonoliposome- |
|---|---|
| (treated with vehicle) | treated mice |
| 16.33 mmHg | 10.25 mmHg |
| 18.66 mmHg | 9.62 mmHg |

1. A method for treating a prostate proliferative disorder in a patient, which method comprises administering arsonoliposomes comprising arsonolipids, wherein the arsonolipids are 2,3-diacyloxypropylarsonic acids, to a patient in need thereof.

2. The method of claim 1, wherein the arsonolipids are racemic 2,3-diacyloxypropylarsonic acids.

3. The method of claim 1, wherein the arsonolipids are optically active 2,3-diacyloxypropylarsonic acids.

4. The method according to claim 1, wherein said arsonolipids have formula I 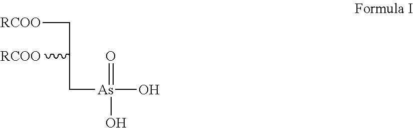
wherein the RCOO acyl chains are saturated linear, saturated branched, unsaturated linear or unsaturated branched acyl chains presenting from 6 to 20 carbon atoms.

5. The method according to claim 4, wherein said RCOO acyl chains are selected from the group consisting of hexanoyl, octanoyl, decanoyl, dodecanoyl, tetradecanoyl, hexadecanoyl and octadecanoyl chains.

6. The method according to claim 1, wherein said arsonoliposomes may further carry polyoxyethylene glycol (PEG) molecules.

7. The method of claim 6, wherein said PEG molecules are conjugated to phospholipids in the arsonoliposomes.

8. The method of claim 7, wherein said phospholipids are selected from the group consisting of
2-distearoyl-sn-glycero-3-phosphoethanolamine (DSPE), dipalmitoyl phosphatidylethanolamine (DPPE), dioleyl-sn-glycero-3-phosphoethanolamine (DOPE), and mixtures thereof.

9. The method according to claim 1, wherein the arsonoliposomes further comprise 1,2-distearoyl-sn-glycero-3-phosphocholine (DSPC) molecules.

10. The method according to claim 1, wherein the arsonoliposomes further comprise 1,2-distearoyl-sn-glycero-3-phosphoethanolanine (DSPE) molecules conjugated with polyoxyethylene glycol (PEG).

11. The method according to claim 1, wherein the arsonoliposomes further comprise 1,2-distearoyl-sn-glycero-3-phosphoethanolanine (DSPE) molecules conjugated with polyoxyethylene glycol (PEG) 2000.

12. The method according to claim 1, wherein the arsonoliposomes further comprise cholesterol molecules.

13. The method of claim 1, wherein the arsonoliposomes contain 1,2-distearoyl-sn-glycero-3-phosphocholine (DSPC), arsonolipids and cholesterol.

14. The method of claim 13, wherein the arsonoliposomes contain 1,2-distearoyl-sn-glycero-3-phosphocholine (DSPC), arsonolipids and cholesterol in molar ratio 12/8/10.

15. The method of claim 14, the arsonoliposomes contain 1,2-distearoyl-sn-glycero-3-phosphocholine (DSPC), arsonolipids and cholesterol in molar ratio 12/8/10, and 8 mol % 1,2-distearoyl-sn-glycero-3-phosphoethanolanine (DSPE) molecules conjugated with polyoxyethylene glycol (PEG) 2000.

16. The method of claim 13, wherein the arsonoliposomes contain 1,2-distearoyl-sn-glycero-3-phosphocholine (DSPC), arsonolipids and cholesterol in molar ratio 17/3/10.

17. The method of claim 1, wherein the patient is human.

18. The method of claim 1, wherein the patient is affected with prostate carcinoma.

19. The method of claim 18, wherein the patient is affected with metastatic prostate carcinoma.

20. The method of claim 1, wherein the patient is affected with begnin prostatic hyperplasia.

21. The method of claim 1, wherein said arsonoliposomes are administered subcutaneously or intramuscularly.

22. The method of claim 1, wherein said arsonoliposomes are administered intravenously.

23. The method of claim 1, wherein said arsonoliposomes are administered directly into or in vicinity to the prostate.

24. The method of claim 1, wherein said arsonoliposomes are administered once a week.
